(19) United States
(12) Patent Application Publication
(10) Pub. No.: US 2022/0007994 A1
(43) Pub. Date: Jan. 13, 2022

(54) PASSIVE HEALTH MONITORING SYSTEM FOR DETECTING SYMPTOMS OF PARKINSON'S DISEASE

(71) Applicant: Goldman Sachs & Co. LLC, New York, NY (US)

(72) Inventor: Michael Mattioli, Staten Island, NY (US)

(21) Appl. No.: 17/484,454

(22) Filed: Sep. 24, 2021

(51) Int. Cl. A61B 5/00

(52) U.S. Cl. CPC A61B 5/4082; A61B 5/681; A61B 5/6828; A61B 5/7465; A61B 5/6815; A61B 5/742; A61B 5/6824

(57) ABSTRACT

A health monitoring system that passively monitors a person for Parkinsonian gait is disclosed. The health monitoring system includes a plurality of user devices. The user devices are typically located on the user due to the user devices performing a primary functionality associated with the user devices. The user devices may perform a secondary function of passively monitoring the person for Parkinsonian gait based on a size of an area formed between the user devices while located on the user's body. The size of the area and how the size of the area changes over time can be used as a heuristic for early detection of Parkinsonian gait.

### BACKGROUND

### 1. Field of Art

The disclosure generally relates to detection of Parkinson's disease, and more specifically to passive detection of Parkinson's disease using user devices of a person.

### 2. Description of the Related Art

Parkinson's disease is a neurodegenerative disorder that mainly affects a person's motor functions. Early symptoms of Parkinson's disease include tremors, micrographia, trouble sleeping, trouble speaking, facial masking, poor posture, and trouble moving and walking. Although there is no known cure for Parkinson's disease, early detection of Parkinson's disease can improve a person's quality of life.

Conventional systems for early detection of Parkinson's disease rely on active data collection to determine whether a person is exhibiting symptoms of Parkinson's disease. During active data collection, a person visits a health care facility where the person undergoes one or more tests to detect whether the person has Parkinson's disease in its early stages. However, active data collection involves the person's conscious interaction during the test. Thus, the person may unknowingly modify his or her behavior during the test thereby masking symptoms of Parkinson's disease. Thus, conventional systems for detecting Parkinson's disease may be inaccurate.

### SUMMARY

A health monitoring system that passively monitors a person for symptoms of Parkinson's disease is disclosed. In one embodiment, the health monitoring system includes a plurality of user devices of a user. The user devices are typically located on the user due to the user devices performing a primary functionality associated with the user devices. Since the user devices are already normally worn by the user, the user devices may also perform a secondary function of passively monitoring the person for Parkinsonian gait which is a symptom of Parkinson's disease. Due to the passive monitoring performed by the user devices, the user does not actively try to modify his or her behavior that would influence the diagnosis of Parkinson's disease.

In one embodiment, at least one of the user devices calculates a size of an area formed between the user devices while located on the user's body. The size of the area and how the size of the area changes over time can be used as a heuristic for early detection of Parkinsonian gait. Responsive to the user exhibiting signs of Parkinsonian gait, at least one of the user devices may display a notification to the user warning the user that he or she is exhibiting symptoms of Parkinson's disease in one embodiment. In another embodiment, one of the user devices may transmit data indicating how the size of the area formed between the user devices changes over time to a health care provider of the user. The health care provider may review the data and determine whether the user should visit the health care provider for further diagnosis of Parkinson's disease.

The features and advantages described in the specification are not all inclusive and, in particular, many additional features and advantages will be apparent to one of ordinary skill in the art in view of the drawings, specification, and claims. Moreover, it should be noted that the language used in the specification has been principally selected for readability and instructional purposes only, and may not have been selected to delineate or circumscribe the inventive subject matter.

### BRIEF DESCRIPTION OF THE DRAWINGS

The disclosed embodiments have advantages and features that will be more readily apparent from the detailed description, the appended claims, and the accompanying figures (or drawings). A brief introduction of the figures is below.

FIG. 1 illustrates a health monitoring environment according to one embodiment.

FIG. 2 illustrates locations of user devices on a user according to one embodiment.

FIG. 3 illustrates a detailed view of a user device according to an embodiment.

FIG. 4A illustrates a region formed between user devices of a person with Parkinsonian gait and FIG. 4B illustrates a region formed between user devices of a person without Parkinsonian gate according to one embodiment.

FIG. 5 illustrates a notification displayed on a user device that the user is exhibiting symptoms of Parkinson's disease according to one embodiment.

FIG. 6 illustrates example data transmitted to a health care provider for further evaluation of Parkinson's disease according to one embodiment.

FIGS. 7A to 7C illustrate interaction diagrams describing a process performed by each user device to calculate an area of a region formed by the user devices according to an embodiment.

FIG. 8 illustrates a method flow diagram describing a process of detecting symptom of Parkinson's disease according to an embodiment.

FIG. 9 is a system diagram of a computer system, according to one embodiment.

### DETAILED DESCRIPTION

The Figures (FIGS.) and the following description relate to embodiments by way of illustration only. It should be noted that from the following discussion, alternative embodiments of the structures and methods disclosed herein will be readily recognized as viable alternatives that may be employed without departing from the principles of what is claimed. Reference will now be made in detail to several embodiments, examples of which are illustrated in the accompanying figures. It is noted that wherever practicable similar or like reference numbers may be used in the figures and may indicate similar or like functionality.

### System Environment

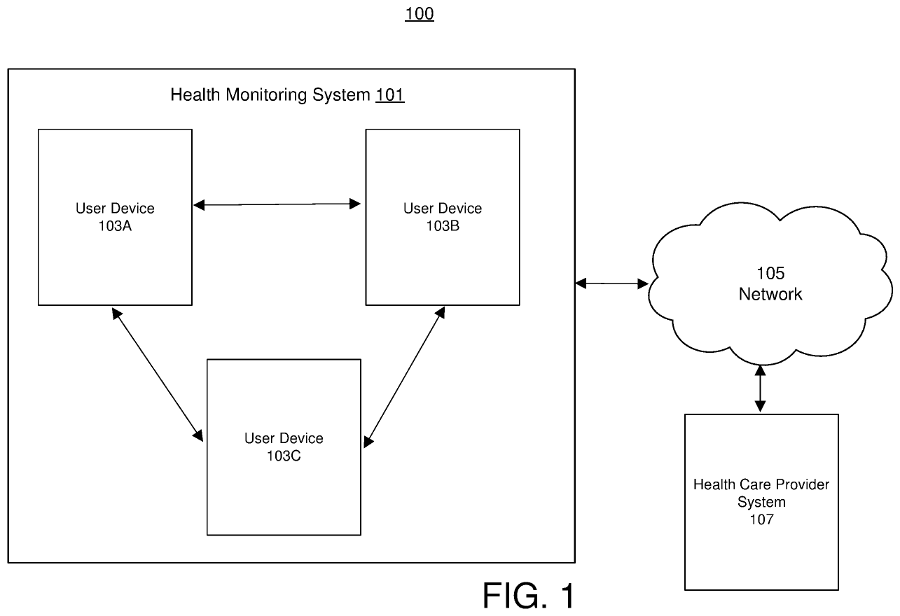

FIG. 1 is a health tracking environment 100 that includes a health monitoring system 101 and a health care provider system 107 according to one embodiment. Generally, the health monitoring system 101 monitors the health of a person (e.g., a user) that is associated with the health monitoring system 101. In one embodiment, the health monitoring system 101 monitors for symptoms of Parkinson's disease. Parkinson's disease can affect the gait of a person as he or she walks. The change in the person's gait is referred to as Parkinson's gait and is a symptom of Parkinson's disease that is monitored (i.e., tracked) by the health monitoring system 101. Responsive to the health monitoring system 101 detecting that the person is exhibiting Parkinson's gait, the health monitoring system 101 may send a notification to the heath care provider system 101 or notify the user via at least one of the user devices. In one embodiment, the health care provider system 101 represents a medical facility (e.g., a hospital) that the person visits for health related visits.

As shown in FIG. 1, the health monitoring system 101 includes a plurality of user devices 103. Specifically, the health monitoring system includes user device 103A, user device 103B, and user device 103C. In one embodiment, the health monitoring system 103 includes at least three user devices 103. However, in other embodiments the health monitoring system 103 may include additional user devices 103 than shown in FIG. 1.

The user devices 103 are representative of mobile consumer electronic devices of the person. The user devices 103 may be different types of mobile consumer electronic devices that each perform a different primary function for the person as well as a secondary function of passively monitoring the user for Parkinsonian gait. Examples of user devices 103 include a smart phone, a smart watch, smart glasses, a headset, earphones etc. Each of the different types of user devices is configured to perform a primary function. For example, the primary function of a smart phone is to make phone calls whereas a primary function of earphones is to listen to audio content.

In one embodiment, users devices 103 included in the health monitoring system 101 all have an inertial measurement unit (IMU) and/or other sensors capable of providing location attributes for each user device such as elevation angle, azimuth angle, velocity, etc. In one embodiment, the user devices 103 included in the health monitoring system 101 also support ultra-wideband (UWB) for communication between the user devices 103. UWB is a radio technology that uses low energy level for short-range, high-bandwidth communications over a large portion of the radio spectrum. Other radio technologies that provide location tracking attributes such as Bluetooth 5.1 may also be used for communication between the user devices 103 in other embodiments.

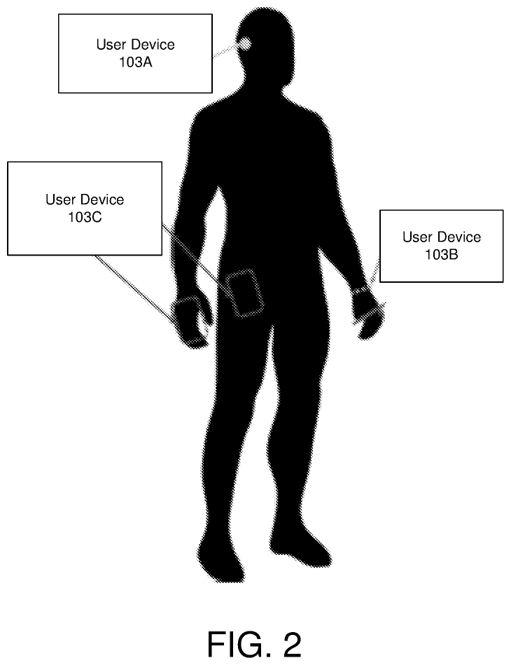

Generally, the user devices 103 are located at different positions on the person at the same time. FIG. 2 illustrates an example of different positions of user devices on the person according to one embodiment. As shown in FIG. 2, user device 103A may be earphones located at the person's ears, user device 103B may be a smart watch located on the person's wrist, and user device 103C may be a smart phone that is proximate the user's hip (e.g., in the person's pocket or hand). The person carries or wears each of the user devices 103 in order to benefit from the primary functionality of each of the user devices 103 such as using the smart phone to make phone calls, using the earphones to listen to audio content such as music, and using the smart watch to view the time and interact with the smart phone.

The health monitoring system 101 leverages the fact that the user normally wears the user devices 103 for their primary functionality and uses the user devices 103 to also perform the secondary functionality of passive monitoring of Parkinsonian gait. For example, the person may normally wear the user devices 103 daily or multiple times every week for the primary function of the user devices 103 thereby allowing the health monitoring system 101 to passively monitor for symptoms of Parkinson's disease. Unlike an active test for Parkinson's disease, the person will not attempt to change his or her behavior while the health monitoring system 101 monitors for Parkinson's disease using user devices 103 since the person uses the user devices 103 for the primary functionality provided by the user devices 103. In one embodiment, the health monitoring system 101 monitors for Parkinson's disease after the person has provided consent.

Referring back to FIG. 1, the health monitoring system 101 is in communication with health care provider system 107 via a network 105. The network 105 provides a communication infrastructure between the health monitoring system 101 and the health care provider system 107. The network 105 is typically the Internet, but may be any network, including but not limited to a Local Area Network (LAN), a Metropolitan Area Network (MAN), a Wide Area Network (WAN), a mobile wired or wireless network, a private network, or a virtual private network.

### User Device

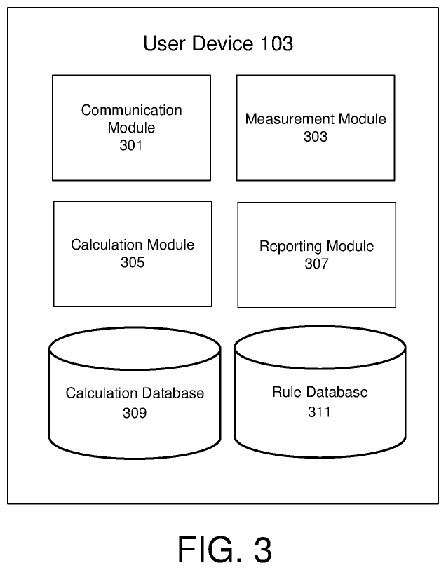

As shown in FIG. 1, the health monitoring system 101 includes a plurality of user devices 103A to 103C. FIG. 3 illustrates a detailed view of each of the user devices 103. In one embodiment, each user device 103 includes a communication module 301, a measurement module 303, a calculation module 305, a reporting module 307, a calculation database 309, and a rule database 311. Note that in other embodiments, the user device 103 may include other modules that shown in FIG. 3.

In one embodiment, each user device 103 may be configured to operate in either a transmitting mode or a receiving mode. During the transmitting mode of a given user device 103, the communication module 301 is configured to operate as a transmitter and transmits a radio frequency RF signal (e.g., a wireless signal) to the other user devices 103 that are operating in the receiving mode. For example, user device 103A operating in a transmitting mode transmits the RF signal that is received by user devices 103B and 103C that are operating in the receiving mode. During the receiving mode of a given user device 103, the communication module 301 is configured to operate as a receiver and receives RF signals from other user devices that are operating in the transmitting mode. For example, user device 103A operating in the receiving mode receives RF signals from user devices 103B and 103C that are operating in the transmitting mode. Each user device 103 may transmit and/or receive RF signals using a wireless communication protocol such as UWB as described above. However, other wireless communication protocols such as Bluetooth 5.1 may be used.

In one embodiment, the measurement module 303 of a given user device 103 measures location attributes of other user devices 103 included in the health monitoring system 101. For example, the measurement module 303 of user device 103A measures location attributes of user devices 103B and 103C, the measurement module 303 of user device 103B measures location attributes of user devices 103A and 103C, and the measurement module 303 of user device 103C measures location attributes of user devices 103A and 103B. In one embodiment, the measurement module 303 of a given user device 103 measures the location attributes of another user device 103 based on the RF signal received from the other user device. For example, user device 103A measures the location attributes of user device 103B based on the RF signal received from user device 103B.

As mentioned previously, the RF signals are transmitted and received using UWB. UWB includes the feature of orientation estimation and the feature of distance estimation. In one embodiment, the estimated location attributes of another user device measured by the measurement module 303 includes the orientation of the RF signals received from the other device 103. The orientation of a RF signal represents the orientation of the other user device 103 that transmitted the RF signal. In one embodiment, the orientation estimation of a RF signal includes an estimation of the Angle-of-Arrival (AoA) of the RF signal transmitted from another user device included in the health monitoring system 101.

In one embodiment, the estimated location attributes of another user device 103 measured by the measurement module 303 includes the distance between the given user device 103 and the other user device 103 that transmitted the RF signal. The measurement module 303 may measure the distance between the given user device 103 and the other user device 103 by measuring the time it takes for the RF signal to pass between the two user devices 103 which provides a more precise distance measurement that a signal-strength based estimation. By knowing the distance between the given user device 103 (e.g., user device 103A) and each of the other user devices 103 (e.g., user devices 103B and 103C) and the orientation of the other user devices, the measurement module 303 can determine the distances between the user devices 103A to 103C.

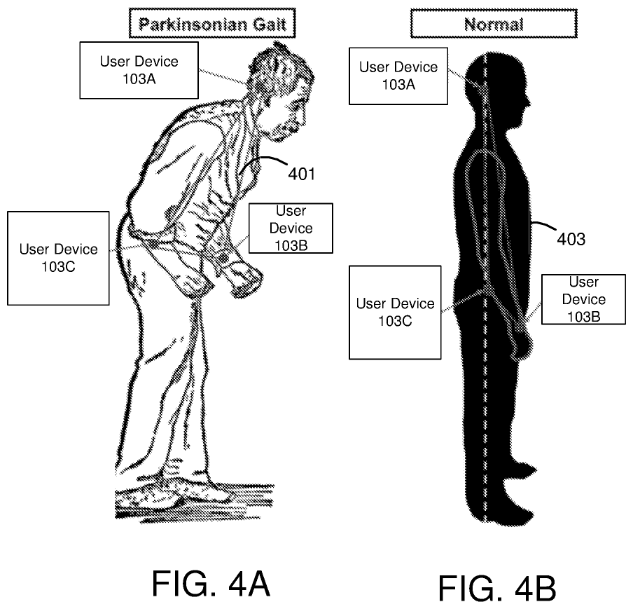

The calculation module 305 calculates a size of a region formed between user devices 103A to 103C. In one embodiment, the region formed between the users devices 103A to 103C while located on the person is a triangle and the size of the region comprises the area of the triangle. FIGS. 4A and 4B illustrate the region formed by user devices 103A to 103C according to one embodiment. Specifically, FIG. 4A illustrates a triangle 401 formed by user devices 103A to 103C located on a person with Parkinsonian gait whereas FIG. 4B illustrates a triangle 403 formed by user devices 103A to 103C located on a person without Parkinson's gait (e.g., a normal gait).

A person with Parkinsonian gait usually takes small, shuffling steps and is hunched over with less movement in the person's arms while walking. Furthermore, the forearms of a person with Parkinsonian gait may be hinged at the elbow forming a 45 degree to 90 degree angle. As a result, the area of the triangle 401 formed by the user devices 103A to 103C that is located on a person with Parkinsonian gait is less than an area of the triangle formed by user devices 103A to 103C that are located on a person without Parkinsonian gait as shown in FIG. 4A. Furthermore, the area of the triangle 401 formed by the user devices 103A to 103C that is located on a person with Parkinsonian gait stays relatively constant over a period of time since the person is hunched over with a relatively fixed arm position.

In contrast, a person without Parkinsonian gait stands upright with their arms at their side as shown in FIG. 4B. As the person walks, the person's arms move forward and backward thereby forming a triangle 403 having a larger area than the triangle 401 formed on a person with Parkinsonian gait. Furthermore, the area of the triangle 403 of a person without Parkinsonian gait changes over time since the person's arms move forward and backward as the person walks.

Referring back to FIG. 3, in one embodiment, the calculation module 305 of a given user device 103 calculates the size of the region (e.g., the triangle) formed by user devices 103A to 103C based on the estimated location attributes of the other user devices 103. As mentioned above, the estimated location attributes for each of the other user devices 103 includes an orientation of the other user device 103 and the distance between the given user device and the other user device 103. From the estimated location attributes, the calculation module 305 calculates the area of the region formed by user devices 103A to 103C. The calculation module 305 stores the calculated area in the calculation database 309.

In one embodiment, the calculation module 305 periodically calculates the area of the region formed by the user devices 103A to 103C and stores the calculated area of the region in calculation database 309. For example, the calculation module 309 continually calculates the area of the region formed by the user devices 103A to 103C responsive to the calculation module 309 determining that the user is walking. The calculation module 309 may determine that the person is walking as a result of IMU in the user device 103 measuring that the user device 103 has a speed between 3 to 4 miles per hour which is the average walking speed for most people. In another example, the calculation module 309 calculates the area of the region formed by the user devices 103A to 103C every second responsive to the calculation module 309 determining that the user is walking.

In one embodiment, the calculation module 309 compares each calculated area of the region formed by the user devices 103A to 103C to a health rule stored in the rule database 311. In one embodiment, the health rule includes an area based rule that specifies an area formed between user devices 103 on a person without Parkinsonian gate. In one embodiment, the rule database 311 may store multiple area based rules where each area based rule is associated with a particular height or height range. Multiple area based rules that are height dependent may be stored since the area of the region formed by user devices 103A to 103C for a person without Parkinsonian gait changes depending on the height of the person. For example, a person who is 5 foot tall may form a region with devices 103A to 103C that is considered “healthy,” but the region is smaller than the area of the region formed by user devices 103A to 103C for a person who is 7 foot tall. In other embodiments, the area based rules may be based on other factors such as age and gender for example.

In one embodiment, the calculation module 305 stores an indication each time the comparison indicates that the calculated area of the region formed by the user devices 103A to 103C is less than the area specified in the health rule. A single instance of the calculated area being less than the area defined in the health rule does not necessarily indicate the person is exhibiting symptoms of Parkinsonian gate. Accordingly, the rule database 311 also stores a time based rule that specifies a threshold number of times that the calculated area violates the area based rule over a particular time period. For example, the time based rule may specify that four violations of the area based rule per day over a time period of 6 to 12 months must occur to result in a warning of Parkinsonian gait. However, other number of violations and time periods may be used.

In one embodiment, the time based rule may also specify an action for the user device 103 to perform responsive to the criteria of the time based rule being satisfied that indicates the symptom of Parkinsonian gait. The action may include for the user device 103 to display a notification to the person that the person is exhibiting symptoms of Parkinson's disease and to contact the person's health care provider system 107. In one embodiment, the action may also indicate to transmit a notification to a user device of another person such as the user's relative or friend in addition to the person exhibiting the symptoms of Parkinson's disease.

In addition to or alternatively to displaying the notification on the user device 103, the action may include and instruction to transmit data including calculated areas of the region formed by the person's user devices 103A to 103C over time to the health care provider system 107. By sending the calculations, the person's health care provider (e.g., a doctor) may review the calculations and determine whether the person should visit the health care provider system 107 for further diagnosis.

In one embodiment, one of the user devices from user devices 103A to 103C is designated as the primary user device and the remaining user devices are designated as a secondary user device. For example, the user device that is a smart phone may be designated the primary device. Each calculation module 305 of the user devices 103A to 103C is configured to calculate the area of the region formed by user devices 103A to 103C at the same instance in time. For each instance in time, the calculation module 305 of the primary user device receives the calculated areas from the remaining secondary user devices. In one embodiment, the calculation module 305 of the primary user device calculates an average of the calculated areas for each instance in time and stores the average in the calculation database 309. The calculation database 309 compares the calculate averages of the area formed by the user devices 103A to 103C to the rules described above to determine whether the person is exhibiting symptoms of Parkinsonian gait.

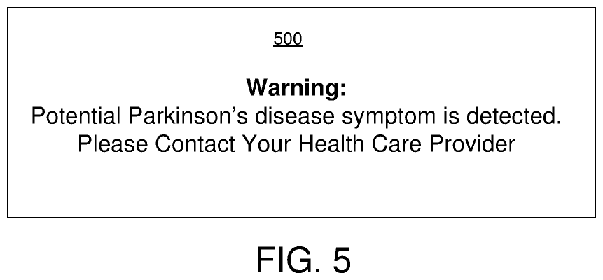

In one embodiment, the reporting module 307 generates reports regarding a person potentially exhibiting symptoms of Parkinson's disease. The reporting module 307 may generate different reports depending on the time based rule that specifies whether the person and/or health care provider is to receive a notification. FIG. 5 illustrates an example of a notification 500 generated by the reporting module 307 for the person exhibiting signs of Parkinson's disease according to one embodiment. The notification 500 may include a warning that a symptom of Parkinson's disease is detected and a recommendation for the person to contact his or her health care provider. A similar notification may be sent to another person that is responsible for the person exhibiting signs of Parkinson's disease, such as a relative, friend, or guardian.

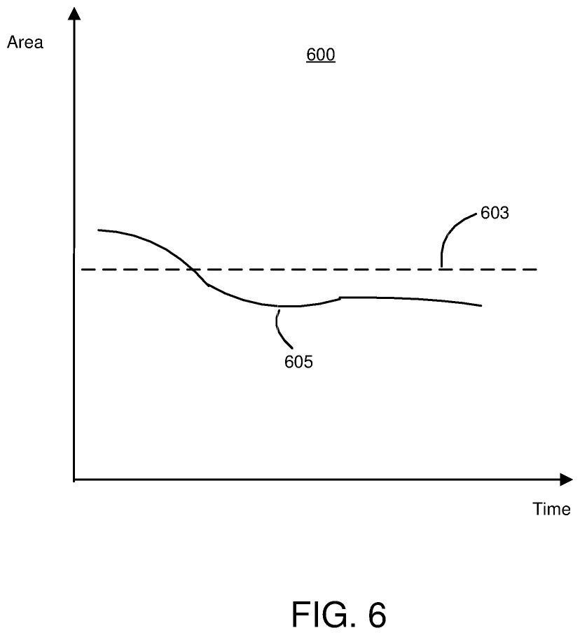

FIG. 6 illustrates an example graph 600 of data indicative of the calculated areas plotted over time. The reporting module 307 may generate the graph based on the calculations stored in the calculation database 309 and transmit the graph to the person's health care provider system 107 for further review. As shown in FIG. 6, the graph 600 may include a threshold 603 that indicates the area formed by user devices 103 that are representative of a person without Parkinsonian gait. In the graph 600, curve 605 represent the calculated areas of the region formed by user devices 103A to 103C over time. As shown in FIG. 6, the calculated areas are below the threshold 603 for a period of time (e.g., 3 months) where the area stays relative constant during the period of time. Since the graph is below the threshold, the graph indicates that the area formed between the user devices is less than the area typically formed in a person without Parkinsonian gait. Furthermore, the graph shows that the area stays relative constant over time under the threshold 603 further indicative that the user's arms are likely in a relatively fixed position while walking which is a symptom of Parkinsonian gait.

### Method Flow Diagrams

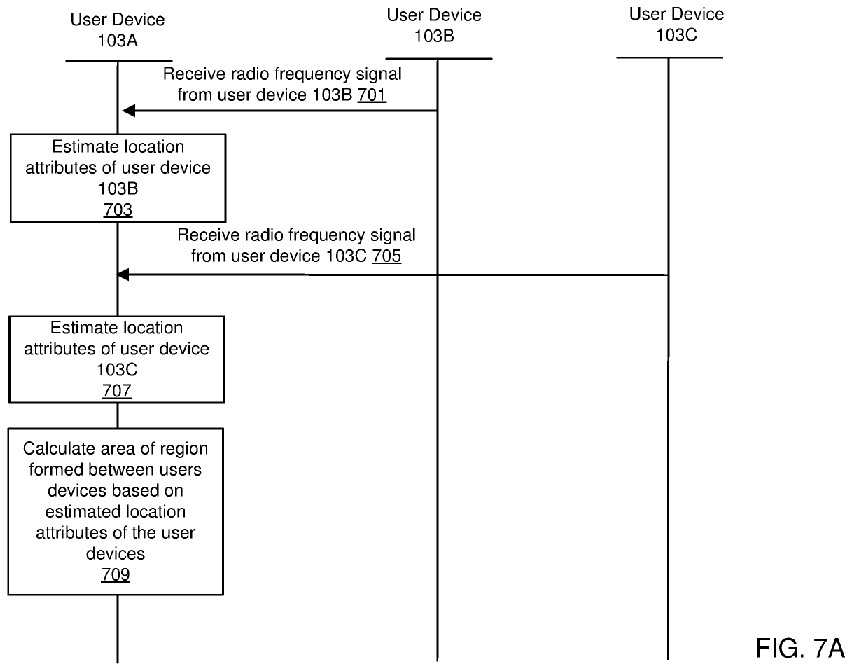

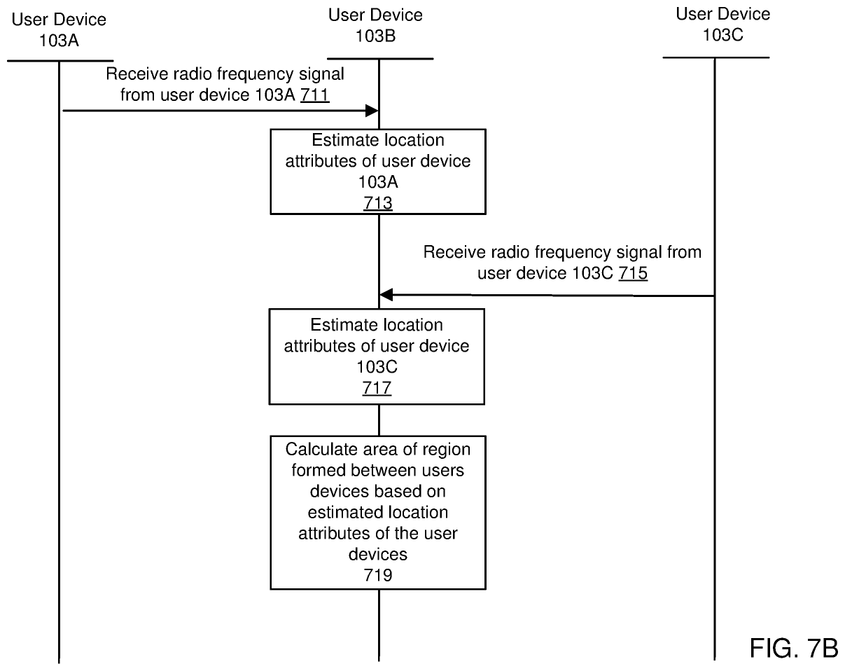

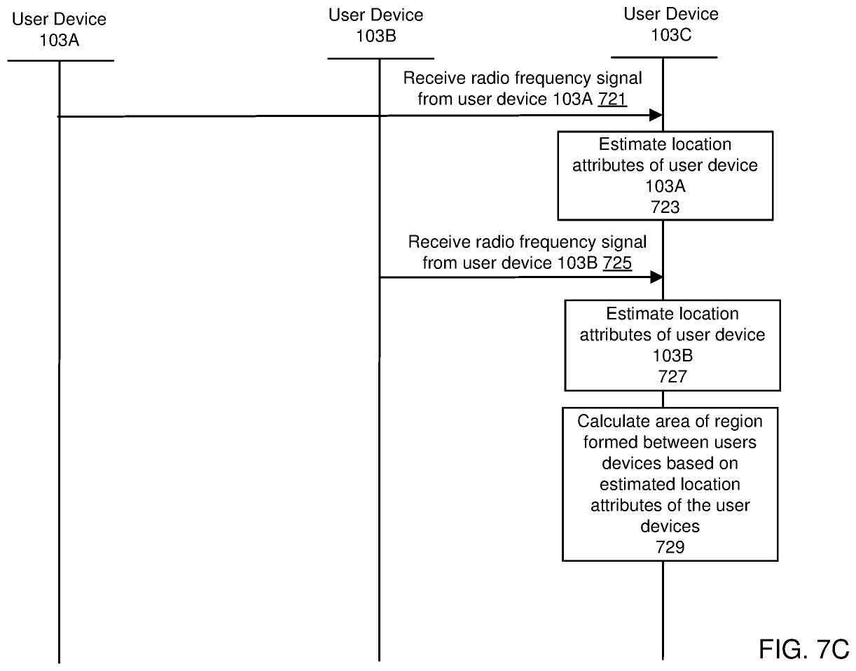

FIGS. 7A to 7C illustrate interaction diagrams describing a process of calculating an area of a region formed by the user devices 103A to 103C according to an embodiment. Note in other embodiments, other steps may be shown than those illustrated in FIG. 7A to 7C.

FIG. 7A illustrates an interaction diagram describing a process of user device 103A to calculate an area of a region formed by the user devices 103A to 103C according to an embodiment. In one embodiment, while the user device 103A is in the receiving mode, the user device 103A receives 701 a RF signal from user device 103B and estimates 703 location attributes of user device 103B based on the received RF signal. While the user device 103A is in the receiving mode, the user device 103A also receives 705 a RF signal from user device 103C and estimates 709 location attributes of user device 103B based on the received RF signal. The user device 103A then calculates 709 an area of the region (e.g., a triangle) formed between user devices 103A to 103C based on the estimated location attributes of the user devices 103B and 103C.

FIG. 7B illustrates an interaction diagram describing a process of user device 103B to calculate an area of a region formed by the user devices 103A to 103C according to an embodiment. In one embodiment, while the user device 103B is in the receiving mode, the user device 103B receives 713 a RF signal from user device 103A and estimates 713 location attributes of user device 103A based on the received RF signal. While the user device 103B is in the receiving mode, the user device 103B also receives 715 a RF signal from user device 103C and estimates 717 location attributes of user device 103C based on the received RF signal. The user device 103B then calculates 719 an area of the region formed between user devices 103A to 103C based on the estimated location attributes of the user devices 103A and 103C.

FIG. 7C illustrates an interaction diagram describing a process of user device 103C to calculate an area of space formed by the user devices 103A to 103C according to an embodiment. In one embodiment, while the user device 103C is in the receiving mode, the user device 103C receives 721 a RF signal from user device 103A and estimates 723 location attributes of user device 103A based on the received RF signal. While the user device 103C is in the receiving mode, the user device 103B also receives 725 a RF signal from user device 103B and estimates 727 location attributes of user device 103B based on the received RF signal. The user device 103C then calculates 729 an area of the region formed between user devices 103A to 103C based on the estimated location attributes of the user devices 103A and 103B.

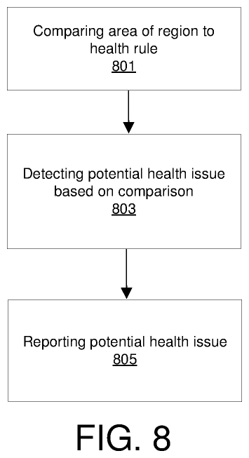

FIG. 8 illustrates a method for determining whether a person is exhibiting symptoms of Parkinson's gait according to one embodiment. The method shown in FIG. 8 is performed by at least one of the user devices 103A to 103C. For example, the primary user device 103A may perform the method shown in FIG. 8 or all of the user devices 103A to 103C may perform the method shown in FIG. 8.

In one embodiment, a user device 103 compares 801 an area of the region calculated by the user device 103 as previously described with respect to FIGS. 7A to 7C to a health rule. The health rule may be an area based rule that indicates a threshold area. Responsive to the area of space violating the health rule, the user device 103 may store an indication of the violation. The user device 103 may compare the total number of violations to a time based health rule. The time based health rule specifies a threshold number of violations over a predetermined time period that is indicative that the person may exhibit signs of Parkinsonian gate.

The user device 103 detects 803 a potential health issue with the person based on the comparison. For example, the user device 103 may determine that the person is exhibiting signs of Parkinsonian gait responsive to the number of violations over the predetermined time period violating the time based health rule. The user device 103 reports 805 the potential health issue. In one embodiment, the report is display on the user device 103 and/or may be transmitted to the person's health care provider.

### Hardware Components

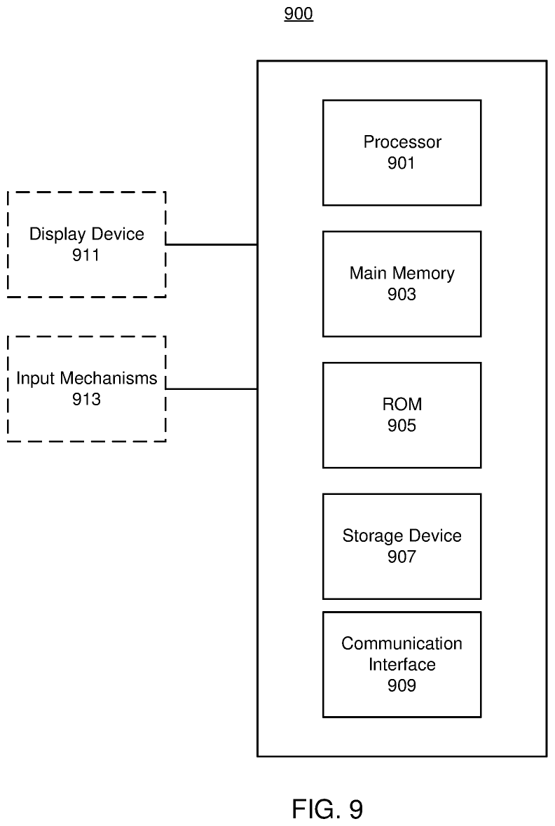

FIG. 9 is a diagram illustrating a computer system 900 upon which embodiments described herein may be implemented within the user devices 103A to 103C and the health care provider system 107. For example, the user devices 103A to 103C and the health care provider system 107 may each be implemented using a computer system such as described by FIG. 9.

In one implementation, the user devices 103A to 103C and the health care provider system 107 each include processing resources 901, main memory 903, read only memory (ROM) 905, storage device 907, and a communication interface 909. The user devices 103A to 103C and the health care provider system 107 each include at least one processor 901 for processing information and a main memory 903, such as a random access memory (RAM) or other dynamic storage device, for storing information and instructions to be executed by the processor 901. In one embodiment, multiple processors may be employed by the user devices 103A to 103C and the health care provider system 107 to perform the techniques described above in order to improve efficiency of the user devices 103A to 103C and the health care provider system 107 and reduce computation time when determining whether a person is exhibiting symptoms of Parkinsonian gait. Main memory 903 also may be used for storing temporary variables or other intermediate information during execution of instructions to be executed by processor 901. The user devices 103A to 103C and the health care provider system 107 may each also include ROM 905 or other static storage device for storing static information and instructions for processor 901. The storage device 907, such as a magnetic disk or optical disk or solid state memory device, is provided for storing information and instructions.

The communication interface 909 can enable each of the user devices 103A to 103C and the health care provider system 107 to communicate with each other through use of a communication link (wireless or wireline). Each of user devices 103A to 103C and the health care provider system 107 can optionally include a display device 911, such as a cathode ray tube (CRT), an LCD monitor, an LED monitor, OLED monitor, a TFT display or a television set, for example, for displaying graphics and information to a user. An input mechanism 913, such as a keyboard that includes alphanumeric keys and other keys, can optionally be coupled to the computer system 900 for communicating information and command selections to processor 901. Other non-limiting, illustrative examples of input mechanisms 913 include a mouse, a trackball, touch-sensitive screen, or cursor direction keys for communicating direction information and command selections to processor 901 and for controlling cursor movement on display device 911.

Examples described herein are related to the use of the user devices 103A to 103C and the health care provider system 107 for implementing the techniques described herein. According to one embodiment, those techniques are performed by each of the user devices 103A to 103C and the health care provider system 107 in response to processor 901 executing one or more sequences of one or more instructions contained in main memory 903. Such instructions may be read into main memory 903 from another machine-readable medium, such as storage device 907. Execution of the sequences of instructions contained in main memory 903 causes processor 901 to perform the process steps described herein. In alternative implementations, hard-wired circuitry may be used in place of or in combination with software instructions to implement examples described herein. Thus, the examples described are not limited to any specific combination of hardware circuitry and software. Furthermore, it has also proven convenient at times, to refer to arrangements of operations as modules, without loss of generality. The described operations and their associated modules may be embodied in software, firmware, hardware, or any combinations thereof.

Reference in the specification to “one embodiment” or to “an embodiment” means that a particular feature, structure, or characteristic is included in at least one embodiment of the disclosure. The appearances of the phrase “in one embodiment” or “a preferred embodiment” in various places in the specification are not necessarily referring to the same embodiment.

Some portions of the above are presented in terms of methods and symbolic representations of operations on data bits within a computer memory. These descriptions and representations are the means used by those skilled in the art to most effectively convey the substance of their work to others skilled in the art. A method is here, and generally, conceived to be a self-consistent sequence of steps (instructions) leading to a desired result. The steps are those requiring physical manipulations of physical quantities. Usually, though not necessarily, these quantities take the form of electrical, magnetic or optical signals capable of being stored, transferred, combined, compared and otherwise manipulated. It is convenient at times, principally for reasons of common usage, to refer to these signals as bits, values, elements, symbols, characters, terms, numbers, or the like. Furthermore, it is also convenient at times, to refer to certain arrangements of steps requiring physical manipulations of physical quantities as modules or code devices, without loss of generality.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “displaying” or “determining” or the like, refer to the action and processes of a computer system, or similar electronic computing device, that manipulates and transforms data represented as physical (electronic) quantities within the computer system memories or registers or other such information storage, transmission or display devices.

Certain aspects disclosed herein include process steps and instructions described herein in the form of a method. It should be noted that the process steps and instructions described herein can be embodied in software, firmware or hardware, and when embodied in software, can be downloaded to reside on and be operated from different platforms used by a variety of operating systems.

The embodiments discussed above also relates to an apparatus for performing the operations herein. This apparatus may be specially constructed for the required purposes, or it may comprise a general-purpose computer selectively activated or reconfigured by a computer program stored in the computer. Such a computer program may be stored in a non-transitory computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability.

The methods and displays presented herein are not inherently related to any particular computer or other apparatus. Various general-purpose systems may also be used with programs in accordance with the teachings herein, or it may prove convenient to construct more specialized apparatus to perform the required method steps. The required structure for a variety of these systems will appear from the description below. In addition, the embodiments are not described with reference to any particular programming language. It will be appreciated that a variety of programming languages may be used to implement the teachings described herein, and any references below to specific languages are provided for disclosure of enablement and best mode.

### ADDITIONAL CONFIGURATION CONSIDERATIONS

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the disclosure. This description should be read to include one or at least one and the singular also includes the plural unless it is obvious that it is meant otherwise.

Upon reading this disclosure, those of skill in the art will appreciate still additional alternative structural and functional designs through the disclosed principles herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

What is claimed is:

1. A health monitoring system comprising:
a plurality of user devices, each of the plurality of user devices having a different device type and configured to be placed at different parts of a user's body,
wherein at least one of the plurality of user devices is configured to detect that the user is exhibiting signs of Parkinsonian gait based on a size of a region formed between the plurality of user devices placed at different parts of the user's body.

2. The health monitoring system of claim 1, wherein the plurality of user devices comprises:
a first user device having a first device type, the first user device configured to be placed at a first location on the user's body;
a second user device having a second device type that is different from the first device type, the second user device configured to be placed at a second location on the user that is different from the first location; and
a third user device having a third device type that is different from the first device type and the second device type, the third user device configured to be placed at a third location on the user that is different from the first location and the second location.

3. The health monitoring system of claim 2, wherein the first device type comprises a smart phone and the first location is proximate a hip of the user, the second device type comprises a smart watch and the second location is a wrist of the user, and the third device type are earphones and the third location is ears of the user.

4. The health monitoring system of claim 1, wherein each of the plurality of user devices is configured to receive a plurality of radio frequency signals from remaining user devices from the plurality of user devices, and estimate location attributes of the remaining user devices based on the plurality of radio frequency signals.

5. The health monitoring system of claim 4, wherein the plurality of radio frequency signals are received using Ultra-wideband (UWB) and the estimated location attributes include an orientation of each of the remaining user devices and a distance between the user device and each remaining user device.

6. The health monitoring system of claim 5, wherein the region formed between the plurality of user devices is a triangle and at least one of the plurality of user devices is configured to calculate the size of the triangle based on the orientation of each of the remaining user devices and the distance between the user device and each remaining user device, wherein the size is an area of the triangle.

7. The health monitoring system of claim 6, wherein the at least one of the plurality of users devices is configured to calculate the size of the triangle by:
calculating a plurality of instances of the area of the triangle formed between the plurality of user devices over a predefined time period; and
comparing each of the plurality of instances of the area to a threshold area;
wherein the at least one of the plurality of user devices is configured to detect that the user is exhibiting signs of Parkinsonian gait responsive to a threshold number of the plurality of instances of the area violating the threshold area over the predetermined period of time based on the comparison.

8. The health monitoring system of claim 7, wherein the at least one of the plurality of users devices is configured calculate the plurality of instances of the area of the triangle by:
receiving, from the remaining user devices, a plurality of other instances of the area of the triangle formed between the plurality of user devices over the predefined time threshold, the plurality of other instances of the area calculated by the remaining user devices, and
wherein each of the calculated plurality of instances of the area of the triangle is an average of the area of the triangle calculated by the at least one of the plurality of user devices and corresponding other instances of the area of the triangle calculated by the remaining user devices.

9. The health monitoring system of claim 1, wherein at least one of the plurality of user devices is configured to display a notification that the user is exhibiting signs of Parkinson's disease.

10. The health monitoring system of claim 7, wherein at least one of the plurality of user devices is configured to transmit the calculated plurality of instances of the area of the triangle to a health care provider of the user for further evaluation by the health care provider.

11. A method of a health monitoring system including a plurality of user devices, each of the plurality of user devices having a different device type and configured to be placed at different parts of a user's body, the method comprising:
receiving, by at least one of the plurality of user devices, a plurality of radio frequency signals from remaining user devices from the plurality of user devices;
estimating, by the at least one of the plurality of user devices, location attributes of the remaining user devices based on the plurality of radio frequency signals;
calculating, by the at least one of the plurality of user devices, a size of a region formed between the plurality of user devices based on the location attributes of the remaining user devices; and
detecting, by the at least one of the plurality of user devices, that the user is exhibiting signs of Parkinsonian gait based on the size of the region formed between the plurality of user devices.

12. The method of claim 11, wherein the plurality of user devices comprises:
a first user device having a first device type, the first user device configured to be placed at a first location on the user's body;
a second user device having a second device type that is different from the first device type, the second user device configured to be placed at a second location on the user that is different from the first location; and
a third user device having a third device type that is different from the first device type and the second device type, the third user device configured to be placed at a third location on the user that is different from the first location and the second location.

13. The method of claim 12, wherein the first device type comprises a smart phone and the first location is proximate a hip of the user, the second device type comprises a smart watch and the second location is a wrist of the user, and the third device type are earphones and the third location is ears of the user.

14. The method of claim 11, wherein the plurality of radio frequency signals are received using Ultra-wideband (UWB) and the estimated location attributes include an orientation of each of the remaining user devices and a distance between the user device and each remaining user device.

15. The method of claim 14, wherein the region formed between the plurality of user devices is a triangle and the size of the triangle is calculated based on the orientation of each of the remaining user devices and the distance between the user device and each remaining user device, wherein the size is an area of the triangle.

16. The method of claim 15, wherein calculating the size of the region comprises:
calculating, by at least one of the plurality of user devices, a plurality of instances of the area of the triangle formed between the plurality of user devices over a predefined time period; and
comparing, by at least one of the plurality of user devices, each of the plurality of instances of the area to a threshold area;
wherein the detection that the user is exhibiting signs of Parkinsonian gait is responsive to a threshold number of the plurality of instances of the area violating the threshold area over the predetermined period of time based on the comparison.

17. The method of claim 16, wherein calculating the plurality of instances of the area of the triangle by:
receiving, from the remaining user devices, a plurality of other instances of the area of the triangle formed between the plurality of user devices over the predefined time threshold, the plurality of other instances of the area calculated by the remaining user devices, and
wherein each of the calculated plurality of instances of the area of the triangle is an average of the area of the triangle calculated by the at least one of the plurality of user devices and corresponding other instances of the area of the triangle calculated by the remaining user devices.

18. The method of claim 11, further comprising:
displaying, by the at least one of the plurality of user devices, a notification that the user is exhibiting signs of Parkinsonian gate.

19. The method of claim 18, further comprising:
transmitting, by the at least one of the plurality of user devices, a notification to a device of another user that is associated with the user, the notification indicating that the user is exhibiting signs of Parkinsonian gate.

20. The method of claim 16, further comprising:
transmitting, by the at least one of the plurality of user devices, the calculated plurality of instances of the area of the triangle to a health care provider of the user.
